(19) United States
(12) Patent Application Publication
(10) Pub. No.: US 2010/0048476 A1
(43) Pub. Date: Feb. 25, 2010

(54) NOVEL USE OF BACTERIOCIN DERIVED FROM ENTEROCOCCUS FAECALIS SL-5

(75) Inventors: Myung Jun CHUNG, Seoul (KR); Jae Gu Seo, Gyeonggi-do (KR)

Correspondence Address: LUCAS & MERCANTI, LLP, 475 PARK AVENUE SOUTH, 15TH FLOOR, NEW YORK, NY, 10016, US

(73) Assignee: CELL BIOTECH CO., LTD, Gyeonggi-do (KR)

(21) Appl. No.: 12/482,093

(22) Filed: Jun. 10, 2009

Related U.S. Application Data
(63) Continuation of application No. PCT/KR2008/005118, filed on Sep. 1, 2008.

(30) Foreign Application Priority Data
Aug. 19, 2008 (KR) 10-2008-0080930

(51) Int. Cl. A61K 38/16; C07K 14/195

(52) U.S. Cl. 514/12; 530/324

(57) ABSTRACT

The present invention relates to novel use of bacteriocin derived from Enterococcus faecalis SL-5. More particularly, the present invention relates to a composition for preventing and treating acne comprising a polypeptide of SEQ ID NO: 1 or SEQ ID NO: 2, which is a bacteriocin derived from Enterococcus faecalis SL-5. Also, it relates to use of the bacteriocin for the manufacture of an agent for preventing or treating acne, and a method for preventing or treating acne comprising administering or applying the bacteriocin to subject in need thereof. The present invention provides a polypeptide of SEQ ID NO: 1 or SEQ ID NO: 2 derived from Enterococcus faecalis SL-5, which shows excellent antibacterial activity against Propionibacterium acnes inducing acne. The inventive polypeptide can inhibit growth of Propionibacterium acnes by having excellent antibacterial activity against them inducing acne. Accordingly, the inventive polypeptide can be effectively used in preventing, treating and improving acne.

### TECHNICAL FIELD

The present invention relates to novel use of bacteriocin derived from Enterococcus faecalis SL-5. More particularly, the present invention relates to a composition for preventing and treating acne comprising a polypeptide of SEQ ID NO: 1 or SEQ ID NO: 2, which is a bacteriocin derived from Enterococcus faecalis SL-5. Also, it relates to use of the bacteriocin for the manufacture of an agent for preventing or treating acne, and a method for preventing or treating acne comprising administering or applying the bacteriocin to subject in need thereof.

### BACKGROUND ART

In general, acne is known to develop as a result of inflammatory response caused by irritations due to free fatty acids generated by the lipase of the anaerobic bacterium Propionibacterium acnes which inhabits the skin and various other low-molecular-weight substances. Oral or topical treatments are used to treat acne. Examples include a sebum secretion inhibitor using antiandrogen, a treatment utilizing the anti-inflammatory actions of steroids and non-steroidal analgesic inflammation drugs, an antibacterial agent or a keratin exfoliator such as resorcinol, sulfur, salicylic acid, benzoyl peroxide, retinoic acid or isotretinoin, an antibiotic such as tetracycline, erythromycin, meclocycline, etc. to inhibit the activity of Propionibacterium acnes, or the like. Recently, azelaic acid is also used to treat acne.

Although the aforesaid treatments are effective in treating acne to some extent, they have the following problems. The hormonal treatments are reportedly associated with side effects such as the inhibition of epidermal growth and the abuse of hormones. The keratin exfoliators such as retinoic acid, benzoyl peroxide, etc. are associated with skin irritation and contact dermatitis due to the exfoliation of the horny layer. The antibiotics such as tetracycline are associated with the problems of antibiotic-resistant bacteria and hyperphotosensitivity. Especially, isotretinoin is known to be highly likely to cause teratogenicity.

Accordingly, there are needs for the development of an acne treatment which provides superior therapeutic effect while being safe for human body.

Lactic acid bacteria are widely distributed in the nature and produce lactic acid by fermenting carbohydrate anaerobically. Because this bacterium inhibits the growth of spoilage microorganisms which proliferate well in neutral to alkaline environments, it has been historically used to preserve food in the East and the West. Particularly, it has been used in processing of milk, dairy products, meats, fermented vegetables, and salted fish. Through various organic acids which are metabolic products of the lactic acid bacteria, these bacteria lower pH and improve food preservation. This is because the organic acids such as lactic acid and acetic acid have bactericidal effect on most microorganisms. In addition to the organic acids, hydrogen peroxide and bacteriocins produced by the bacteria are related with preservability.

Bacteriocins are proteins or proteinaceous toxins produced by bacteria to inhibit the growth of phylogenically similar or closely related bacterial strains. They are natural antibacterial proteins distinguished from other antibiotics in that they exhibit narrow antibacterial spectrum and are synthesized directly from the genes of the corresponding bacteria. Bacteriocin is easily found in various fermented foods and is produced by lactic acid bacteria which are taken in through daily diets. Because they are composed of proteins, differently from other antibiotics, they are broken down by proteases in the digestive organ as soon as they are taken in. Because of the non-toxicity and non-residence resulting therefrom, bacteriocins are highly valued as preservative or microorganism control agent for fermented foods. Nisin, which is the best known bacteriocin produced by lactic acid bacteria, has been long used as a food preservative because of its wide antibacterial spectrum. In 1988, the U.S. Food and Drug Administration (FDA) approved nisin as a GRAS (generally recognized as safe) food additive.

### DISCLOSURE

### Technical Problem

Therefore, the inventors of the present invention have conducted intensive studies to search antibiotic activity of Enterococcus faecalis SL-5. Finally, we have found that it has excellent antibacterial activity against Propionibacterium acnes inducing acne and confirmed that a polypeptide of SEQ ID NO: 1 or SEQ ID NO: 2 has excellent antibacterial activity against Propionibacterium acnes. Therefore, we have developed a composition for preventing and treating acne comprising a polypeptide of SEQ ID NO: 1 or SEQ ID NO: 2 as an effective component, thereby completing the present invention.

Accordingly, an objection of the present invention is to provide novel use of a polypeptide of SEQ ID NO: 1 or SEQ ID NO: 2.

### Technical Solution

To achieve the above object, the present invention provides a composition for preventing and treating acne comprising a polypeptide having an amino acid sequence represented by SEQ ID NO: 1 or SEQ ID NO: 2 as an effective component.

In order to accomplish another object, the present invention provides an antibiotic composition against Propionibacterium acnes comprising a polypeptide having an amino acid sequence represented by SEQ ID NO: 1 or SEQ ID NO: 2 as an effective component.

In order to accomplish another object, the present invention provides a method for inhibiting growth of Propionibacterium acnes comprising the step of applying a polypeptide having an amino acid sequence represented by SEQ ID NO: 1 or SEQ ID NO: 2.

In order to accomplish another object, the present invention provides an use of a polypeptide having the amino acid sequence represented by SEQ ID NO: 1 or SEQ ID NO: 2 for the manufacture of an agent for treating acne.

In order to accomplish another object, the present invention provides a method for preventing or treating acne comprising administering or applying a polypeptide having the amino acid sequence represented by SEQ ID NO: 1 or SEQ ID NO: 2 with an effective amount to subject in need thereof.

In order to accomplish another object, the present invention provides an use of a polypeptide having the amino acid sequence represented by SEQ ID NO: 1 or SEQ ID NO: 2 for the manufacture of an antibacterial agent for Propionibacterium acnes.

In order to accomplish another object, the present invention provides a method for removing Propionibacterium acnes comprising administering or applying a polypeptide having the amino acid sequence represented by SEQ ID NO: 1 or SEQ ID NO: 2 with an effective amount to subject in need thereof.

### DESCRIPTION OF DRAWINGS

The above and other objects, features and other advantages of the present invention will be more clearly understood from the following detailed description taken in conjunction with the accompanying drawings, in which:

FIG. 1 shows the antibacterial activity of Enterococcus faecalis SL-5 (A: Propionibacterium acnes ATCC 29399; B: Bacillus subtilis KFRI179; C: Bacillus cereus KCTC 3624; D: Staphylococcus aureus KCTC 1927); and

FIG. 2 shows the result of partial purification of the inventive bacteriocin (A) and the antibacterial activity of a gel replicated therefrom against Propionibacterium acnes (B).

### BEST MODE

Hereinafter, the present invention will be described in detail.

The inventive composition is characterized in that it comprises a polypeptide having the amino acid sequence represented by SEQ ID NO: 1 or SEQ ID NO: 2 as an effective component.

According to an embodiment of the present invention, the inventors of the present invention confirmed an antibiotic activity spectrum of Enterococcus faecalis SL-5. Also, the present invention confirmed that it has antibacterial activity against Propionibacterium acnes inducing acne.

Additionally, according to another embodiment of the present invention, the inventors of the present invention confirmed N-terminal sequences by partial-purifying materials having antibacterial activity, thereby separating peptides having antibacterial activity by PCR. Accordingly, the inventors of the present invention confirmed a polypeptide having the amino acid sequence represented by SEQ ID NO: 1 or SEQ ID NO: 2.

The inventive polypeptide can be chemically synthesized according to any technique known in the art (Creighton, Proteins: Structures and Molecular Principles, W.H. Freeman and Co., NY, 1983). Namely, the inventive polypeptide can be prepared by conventional step-wise liquid or solid phase synthesis, fragment condensation, F-MOC or T-BOC chemistry (Chemical Approaches to the Synthesis of Peptides and Proteins, Williams et al., Eds., CRC Press, Boca Raton Fla., 1997; A Practical Approach, Atherton & Sheppard, Eds., IRL Press, Oxford, England, 1989).

Alternatively, the polypeptide according to the present invention can be prepared by a genetic engineering method. A DNA sequence encoding the polypeptide is first constructed according to any conventional method. The DNA sequence may synthesized by performing PCR using suitable primers. Alternatively, the DNA sequence may also be synthesized by a standard method known in the art, for example using an automatic DNA synthesizer (commercially available from Biosearch or Applied Biosystems). The constructed DNA sequence is inserted into a vector comprising at least one expression control sequence (ex: promoter, enhancer) that is operatively linked to the DNA sequence so as to control the expression of the DNA molecule, and host cells are transformed with the resulting recombinant expression vector. The transformed cells are cultured in a medium and condition suitable to express the DNA sequence, and a substantially pure polypeptide encoded by the DNA sequence is collected from the culture medium. The collection of the pure polypeptide may be performed using a method known in the art, for example, chromatography. In this regard, the term “substantially pure polypeptide” means the inventive polypeptide that does not substantially contain any other proteins derived from host cells. For the genetic engineering method for synthesizing the inventive polypeptide, the reader may refer to the following literatures: Maniatis et al., Molecular Cloning: A Laboratory Manual, Cold Spring Harbor Laboratory 1982; Sambrook et al., supra; Gene Expression Technology, Method in Enzymology, Genetics and Molecular Biology, Method in Enzymology, Guthrie & Fink (eds.), Academic Press, San Diego, Calif. 1991; and Hitzeman et al., J. Biol. Chem., 255, 12073-12080 1990.

Meanwhile, the present invention provides an antibiotic composition against Propionibacterium acnes comprising a polypeptide having an amino acid sequence represented by SEQ ID NO: 1 or SEQ ID NO: 2 as an effective component because the polypeptides inhibit growth of Propionibacterium acnes.

Also, the inventive composition for preventing and treating acne comprising a polypeptide having an amino acid sequence represented by SEQ ID NO: 1 or SEQ ID NO: 2 as an effective component, may be provided in types of product. The product may be cosmetics or medicines.

In case of the medicine, the inventive composition may comprise at least one pharmaceutically acceptable carrier, excipient or diluent additionally. A pharmaceutically acceptable carrier, for example, carriers for the parenteral preparations may be included additionally. The carriers for the parenteral preparations may comprise water, suitable oil, saline, aqueous glucose and glycol, and stabilizers and preservatives additionally. The examples of the stabilizers may be antioxidant such as sodium hydrogen sulfite, sodium sulfite, and ascorbic acid. The examples of the preservatives may be benzalkonium chloride, methyl- or propyl-paraben, and chlorobutanol. The list of pharmaceutically acceptable carriers are disclosed in Remington's Pharmaceutical Sciences, 19th ed., Mack Publishing Company, Easton, Pa., 1995.

A medicine comprising the composition of the present invention may be administered to mammals including human beings by any methods. Preferably, the composition of the present invention may be administered by intracutaneous administration. The term ‘intracutaneous administration’ as used herein means that the composition of the present invention is administrated in cell or skin, thereby transmitting active ingredients. For example, the composition of the present invention may be formulated into a formulation for injection, which is administered by lightly pricking the skin with 30 gauge injection needle. Otherwise, such formulations for injection may be applied directly onto the skin.

The inventive pharmaceutical composition may be formulated according to the administration route such as the above. In case of the parenteral preparations, they can be formulated in the form of injections, creams, lotions, external ointments, oils, moisturizers, gels, aerosols, and nasal inhalers by any method known in the art. These preparations are described in the following formulary known to all pharmaceutical chemists: Remington's Pharmaceutical Science, 15th Edition, 1975, Mack Publishing Company, Easton, Pa. 18042, Chapter 87: Blaug, Seymour.

The total effective amount of the inventive peptide can be administered to a subject as a single dose, or can be administered using a fractionated treatment protocol, in which the multiple doses are administered over a more prolonged period of time. The content of the active ingredient in the inventive composition may be varied depending on the severity of disease. The effective dose of the polypeptide may be determined depending on many factors, such as the age, body weight, health condition, sex, disease severity, diet and excretion of a subject in need of treatment, as well as administration time and administration route. In view of these factors, any person skilled in the art may determine an effective dose. Preferably, the effective dose is 0.001 μg/kg (body weight) to 10 mg/kg (body weight). No particular limitation is imposed on the formulation, administration route and administration mode, as long as a product comprising the inventive composition shows the effects of the present invention.

And, in case of the cosmetics, the inventive polypeptide may be prepared into any formulation adequate for topical application using a method known in the art by additionally including a cosmetically and/or dermatologically acceptable excipient. For example, although not limited thereto, the inventive cosmetic composition may be prepared into solution, gel, solid, emulsion, suspension, microemulsion, microcapsule, microgranule, ionic and/or non-ionic follicular dispersion, cream, lotion, powder, ointment, spray or stick.

The cosmetically and/or dermatologically acceptable excipient may comprise a skin softener, an emulsifier, a thickener and a solvent.

The skin softener is used to soften, soothe, coat, smoothen or moisturize the skin. In the present invention, the skin softener may be any one known in the art. For example, it may be a petroleum based product, a fatty acid ester, an alkyl ethoxylate, a fatty acid ester ethoxylate, a fatty alcohol, a polysiloxane or a mixture thereof. Further, commonly used skin softeners such as propylene glycol, butylene glycol, glycerin, triethylene glycol, spermaceti, wax, fatty acid, fatty alcohol ether, glyceride, acetoglyceride, ethoxylated glyceride, polyhydroxy alcohols and other fatty acid esters, lanolin and its derivatives are included.

The emulsifier serves to make the water phase and the oil phase of the inventive cosmetic composition miscible with each other. In the present invention, one or more of the emulsifier may be included. The emulsifier may be non-ionic, anionic or cationic. The type of the emulsifier may be determined depending on whether the emulsion is water-in-oil type or oil-in-water type. Examples of the suitable emulsifier include, although not limited thereto, sorbitan trioleate, sorbitan tristearate, glycerol monooleate, glycerol monostearate, glycerol monolaurate, sorbitan sesquioleate, sorbitan monooleate, sorbitan monostearate, polyoxyethylene stearyl ether, polyoxyethylene sorbitol beeswax derivative, PEG 200 dilaurate, PEG 200 monostearate, PEG 400 dioleate, sorbitan monopalmitate, polyoxyethylene monostearate, polyoxyethylene sorbitan monostearate, and the like.

The thickener may include crosslinked carboxypolymethylene polymer, ethyl cellulose, polyethylene glycol, gum tragacanth, gum karaya, xanthan gum, bentonite, hydroxyethyl cellulose and hydroxypropyl cellulose.

The solvent may include purified water, alcohol or a mixture of purified water and alcohol.

In addition, other commonly used cosmetic additives including, although not limited thereto, a preservative such as p-hydroxybenzoate ester, an antioxidant such as butylhydroxytoluene, ascorbic acid and its derivative, and tocopherol and its derivative, a wetting agent such as glycerol, sorbitol, 2-pyrrolidone-5-carboxylate, dibutylphthalate, gelatin and polyethylene glycol, a pH buffering agent such as acetate, phosphate, citrate, triethanolamine and carbonate, a wax such as beeswax and paraffin, a viscosifier, an activity enhancer, a colorant, and a fragrance.

Meanwhile, the present invention provides a method for inhibiting growth of Propionibacterium acnes comprising the step of applying a polypeptide having an amino acid sequence represented by SEQ ID NO: 1 or SEQ ID NO: 2 because the polypeptide inhibit growth of Propionibacterium acnes.

Moreover, the present invention provides an use of a polypeptide having the amino acid sequence represented by SEQ ID NO: 1 or SEQ ID NO: 2 for the manufacture of an agent for treating acne. Also, the present invention provides an use of a polypeptide having the amino acid sequence represented by SEQ ID NO: 1 or SEQ ID NO: 2 for the manufacture of an antibacterial agent for Propionibacterium acnes.

The present invention provides a method for preventing or treating acne comprising administering or applying a polypeptide having the amino acid sequence represented by SEQ ID NO: 1 or SEQ ID NO: 2 with an effective amount to subject in need thereof. Also, the present invention provides a method for removing Propionibacterium acnes comprising administering or applying a polypeptide having the amino acid sequence represented by SEQ ID NO: 1 or SEQ ID NO: 2 with an effective amount to subject in need thereof.

The polypeptide having the amino acid sequence represented by SEQ ID NO: 1 or SEQ ID NO: 2 and the effects thereof are described above.

As used herein, the term “effective amount” refers to an amount effective in preventing and treating acne and removing Propionibacterium acnes in subject in need of the inventive polypeptide.

As used herein, the term “subject” means mammals, and particularly animals including human beings. The subject may be human beings in need of preventing and treating acne and removing Propionibacterium acnes

The inventive polypeptide may be administered until the desired effect of the above effects. The inventive polypeptide may be administered by various oral or parenteral routes according to any method known in the art

### ADVANTAGEOUS EFFECTS

Accordingly, the present invention provides a polypeptide of SEQ ID NO: 1 or SEQ ID NO: 2 derived from Enterococcus faecalis SL-5, which shows excellent antibacterial activity against Propionibacterium acnes inducing acne. The inventive polypeptide can inhibit growth of Propionibacterium acnes by having excellent antibacterial activity against them inducing acne. Accordingly, the inventive polypeptide can be effectively used in preventing, treating and improving acne

### MODE FOR INVENTION

Hereinafter, the present invention will be described in detail by examples.

It is to be understood, however, that these examples are for illustrative purpose only and are not construed to limit the scope of the present invention.

### Example 1

### Cell Culturing and Confirmation of Antibacterial Spectrum

1-1. Cell Culturing

Enterococcus faecalis SL-5 (16s rRNA thereof was registered as GenBank Accession No. AY692453) was inoculated to a liquid MRS medium, and cultured for about 16 hours at 37° C., under nitrogen-replaced anaerobic condition. Propionibacterium acnes (ATCC 29399) was cultured in RCM broth (reinforced clostridial medium broth; Merck, Darmstadt, Germany), and Bacillus subtilis (KFRI179), Bacillus cereus (KCTC 3624), Staphylococcus aureus (KCTC 1972), E. coli O157, Listeria monocytogenes, Salmonella paratyphi A and Shigella flexneri were cultured in BHI broth (brain heart infusion broth), at 37° C. for about 16 hours.

1-2. Confirmation of Antibacterial Activity Spectrum of Enterococcus faecalis SL-5

After removing the cellular elements from the culture medium of E. faecalis SL-5 by centrifugation (9,940×g, 15 minutes, 4° C.), the resultant supernatant of the culture medium was further filtered (membrane pore size=0.45 μm) to completely remove the remaining cellular elements. Thus the obtained supernatant portion was named “culture supernatant” Antibacterial activity of the culture supernatant was measured by the “spot-on-the-lawn” method (Van Reenen et al., 2006, Appl. Environ. Microbiol. 72: 7644-7651). The culture supernatant was serially diluted two-folds using oxygen-free water (42.3 mM Na₂HPO₄, 33.1 mM KH₂PO₄, 0.05% (v/v) Tween 80, 3.2 mM L-cystein.HCl, pH 7.0). 10 μL of the diluted extract was dropped on an MRS agar plate containing test bacteria and culturing was carried out at 37° C. Antibacterial activity was identified by determining whether a clear zone was formed around the dropping position.

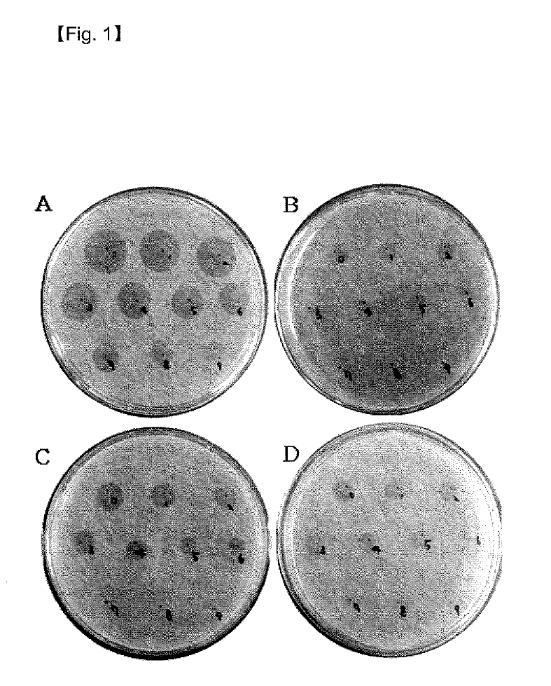

As seen from FIG. 1 and Table 1, Enterococcus faecalis SL-5 had antibacterial activity against the Gram-positive bacteria Bacillus cereus KCTC 3624, Bacillus subtilis KFRI179, Listeria monocytogenes, Propionibacterium acnes ATCC 29399 and Staphylococcus aureus KCTC 1927, but no antibacterial activity against E. coli O157, Shigella flexneri and Salmonella paratyphi A.

TABLE 1

| Bacteria | Inhibition |
|---|---|
| Bacillus cereus KCTC 3624 | + |
| Bacillus subtilis KFRI 179 | + |
| Escherichia coli O157 | − |
| Listeria monocytogenes | + |
| Propionibacterium acnes ATCC 29399 | + |
| Salmonella paratyphi A | − |
| Shigella flexneri | − |
| Staphylococcus aureus KCTC 1927 | + |

### Example 2

### Identification of Antibacterial Substance

2-1. Partial Purification of Antibacterial Substance

Partial purification was carried out in order to identify the substance that exhibit antibacterial activity. Enterococcus faecalis SL-5 was inoculated in 100 mL of MRS medium. After culturing at 37° C. for 16 hours and inoculating in 2 L of fresh MRS medium, Enterococcus faecalis SL-5 was cultivated for 8 hours at 37° C. to prepare a culture supernatant as in Example 1.

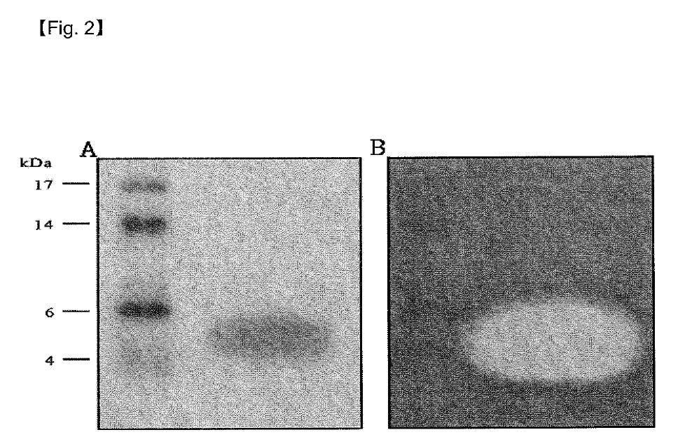

For fractionation of the culture supernatant, ammonium sulfate was slowly added to a final concentration of 70%. After dissolving, centrifugation (20,000×g/30 minutes/4° C.) was carried out to obtain a precipitate. The precipitate was dissolved in a standard buffer solution (50 mM Tris-HCl, pH 7.0) to a final volume of 70 mL. Then, dialysis was carried out against the same buffer with buffer exchange for a total of three changes over 48 hour period, in order to remove the salts included in the solution, using dialysis tubing (MW cut-off=500 dalton). Ultrafiltration (MW cut-off=3,000 dalton) was carried out to concentrate the dialysis solution, and 8 mL of the concentrate (7 mg/mL) was fractionated using a CM Sepharose™ Fast Flow column (GE Healthcare), which is a type of liquid chromatography column. Fractionation was carried out with NaCl concentration gradients from 0 M to 1 M. Antibacterial activity against Propionibacterium acnes was detected at around 0.5 M NaCl. Of the fractions having antibacterial activity, the fraction with the highest activity was analyzed through 16% SDS-tricine gel electrophoresis. As seen in FIG. 2A, most of the proteins were distributed near 5 kDa.

2-2. Identification of Antibacterial Active Substance

In order to confirm whether the protein with a size of about 5 kDa had antibacterial activity against Propionibacterium acnes, activity staining test was carried out as in Example 1-2, using a duplicate gel of the gel used in FIG. 2A.

As seen in FIG. 2B, a clear zone was formed at the portion corresponding to the size of 5 kDa. Therefore, it was confirmed that the protein has antibacterial activity against Propionibacterium acnes.

2-3. Sequence Analysis of Antibacterial Active Substance

In order to determine the amino acid sequence of the protein identified in Example 2-2, the 5 kDa protein was transferred to a PVDF (polyvinylidene fluoride) membrane. After cutting out the protein band showing antibacterial activity from the PVDF membrane, the first ten N-terminal amino acid residues were determined using an automatic protein sequence analyzer.

The N-terminal sequence of the protein was identified as Met-Gly-Ala-Ile-Ala-Lys-Leu-Val-Ala-Lys.

Thus the determined amino acid sequence was compared with protein sequences held in the NCBI (National Center for Biotechnology Information) protein database, using BLAST (Basic Alignment Search Tool). 100% identity was confirmed with enterocin MR10A and B (Martin-Platero et al., 2006, Appl. Environ. Microbiol., 72:4245-4249) and enterocin L50A (Cintas et al., 1998, J. Bacteriol. 180: 1988-1994), and 90% identity was confirmed with enterocin L50B (Cintas et al., 1998, J. Bacteriol. 180: 1988-1994). Because enterocin MR10A and B are considered as variants of enterocin L50, PCR primers of SEQ ID NO: 6 (5′-ATGGGAGCAATCGCAAAATTAGTA-3′) and SEQ ID NO: 7 (5′-TTAATGTCTTTTTAGCCATTTTTCAATTTGATCTATTGT-3′) were synthesized based on the base sequence of enterocin L50, and PCR was carried out using the total DNA of Enterococcus faecalis SL-5 as template. A 287 bp PCR product was obtained. The total base sequence (SEQ ID NO: 5) was determined based on the DNA base sequence of the PCR product. Through alignment comparison of thus determined base sequence with enterocin MR10 and enterocin L50, 100% identity was confirmed with the open reading frame (ORF) of enterocin MR10. Structurally, the gene of Enterococcus faecalis SL-5 has two ORFs in tandem, which were named as SEQ ID NO: 3 and SEQ ID NO: 4.

To conclude, the bacteriocin of Enterococcus faecalis SL-5 according to the present invention is identical to or has substantial identity to enterocin MR10 or L50 however, antibacterial activity of the bacterocin against acne-causing Propionibacterium acnes was newly discovered by the present inventor.

### INDUSTRIAL APPLICABILITY

As can be seen from the foregoing, the present invention provides Enterococcus faecalis SL-5 and a polypeptide of SEQ ID NO: 1 or SEQ ID NO: 2 derived from it, which shows excellent antibacterial activity against Propionibacterium acnes inducing acne. Accordingly, the inventive polypeptide and Enterococcus faecalis SL-5 can inhibit growth of Propionibacterium acnes by having excellent antibacterial activity against them inducing acne. Accordingly, the inventive polypeptide and Enterococcus faecalis SL-5 can be effectively used in preventing, treating and improving acne.

1. A polypeptide comprising an amino acid sequence represented by SEQ ID NO: 1 or SEQ ID NO: 2.

2. The polypeptide of claim 1, wherein the polypeptide has an antibiotic activity against Propionibacterium acnes.

3. The polypeptide of claim 1, wherein the polypeptide treats acne.

4. The polypeptide of claim 1, wherein the polypeptide is part of a cosmetic or medicinal formulation.

5. A method for inhibiting growth of Propionibacterium acnes comprising contacting Propionibacterium acnes with a polypeptide having an amino acid sequence represented by SEQ ID NO: 1 or SEQ ID NO: 2.

6. (canceled)

7. A method for preventing or treating acne comprising administering or applying an effective amount of a polypeptide having the amino acid sequence represented by SEQ ID NO: 1 or SEQ ID NO: 2 to a subject in need thereof.

8-9. (canceled)


SEQUENCE LISTING (7 sequences)

SEQ ID NO: 1 - protein, 44 aa, Enterococcus faecalis SL-5
MGAIAKLVAKFGWPIVKKYYKQIMQFIGEGWAINKIIDWIKKHI

SEQ ID NO: 2 - protein, 43 aa, Enterococcus faecalis SL-5
MGAIAKLVAKFGWPFIKKFYKQIMQFIGQGWTIDQIEKWLKRH

SEQ ID NO: 3 - DNA, 135 bp, Enterococcus faecalis SL-5
atgggagcaatcgcaaaattagtagcaaagtttggatggccaattgttaaaaagtattacaaacaaattatgcaatttattggagaaggatgggcaattaacaaaattattgattggatcaaaaaacatatttaa

SEQ ID NO: 4 - DNA, 132 bp, Enterococcus faecalis SL-5
atgggagcaatcgcaaaattagtagcaaagtttggatggccatttattaaaaaattctacaaacaaattatgcagtttatcggacaaggatggacaatagatcaaattgaaaaatggctaaaaagacattaa

SEQ ID NO: 5 - DNA, 287 bp, Enterococcus faecalis SL-5
atgggagcaatcgcaaaattagtagcaaagtttggatggccaattgttaaaaagtattacaaacaaattatgcaatttattggagaaggatgggcaattaacaaaattattgattggatcaaaaaacatatttaaaaataaggatgtgttaatgtatgggagcaatcgcaaaattagtagcaaagtttggatggccatttattaaaaaattctacaaacaaattatgcagtttatcggacaaggatggacaatagatcaaattgaaaaatggctaaaaagacattaa

SEQ ID NO: 6 - DNA, 24 bp, Artificial Sequence
  primer 1
atgggagcaatcgcaaaattagta

SEQ ID NO: 7 - DNA, 39 bp, Artificial Sequence
  primer 2
ttaatgtctttttagccatttttcaatttgatctattgt